(19) United States
(12) Patent Application Publication
(10) Pub. No.: US 2025/0134993 A1
(43) Pub. Date: May 1, 2025

(54) GLYCATED CHITOSANS FOR TREATMENT OF VIRAL INFECTIONS

(71) Applicant: Immunophotonics, Inc., St. Louis, MO (US)

(72) Inventors: Siu Kit Lam, St. Peters, MO (US); Tomas Hode, St. Louis, MO (US); Luciano Alleruzzo, Des Peres, MO (US); Joseph Raker, Whitesboro, NY (US)

(21) Appl. No.: 18/686,650

(22) PCT Filed: Aug. 26, 2022
(86) PCT No.: PCT/US2022/041680

§ 371 Date: Feb. 26, 2024

(51) Int. Cl. A61K 39/39; A61K 31/722; A61K 39/00; A61K 39/12; A61K 39/125; A61K 39/145; A61K 39/215; A61K 39/235; A61P 37/04

(52) U.S. Cl. CPC A61K 39/39; A61K 31/722; A61K 39/12; A61K 39/125; A61K 39/145; A61K 39/215; A61K 39/235; A61P 37/04; A61K 2039/543; A61K 2039/55583

(57) ABSTRACT

Methods of treating, preventing and/or inhibiting one or more of the symptoms of a respiratory viral infection in a subject by administering a therapeutically effective amount of a glycated chitosan (GC) polymer with characteristics as disclosed herein or a vaccine composition comprising such GC polymer in combination with one or more viral antigens are provided. Methods of reducing morbidity and/or mortality of a respiratory viral infection in a subject, methods of inducing an innate immune response in mucosa of a subject, methods of generating mucosal secretory IgA antibodies and/or neutralizing serum IgG antibodies in a subject, and methods of vaccinating a subject against a respiratory viral infection, wherein these methods comprise administering to the subject a therapeutically effective amount of a glycated chitosan (GC) polymer as disclosed herein or a vaccine composition comprising such GC polymer in combination with one or more viral antigens are further provided.

### FIELD OF THE INVENTION

The present disclosure generally relates to methods of treating, preventing and/or inhibiting one or more of the symptoms and/or reducing morbidity and/or mortality of a respiratory viral infection in a subject, methods of inducing an innate immune response in mucosa of a subject, methods of generating mucosal secretory IgA antibodies and/or neutralizing serum IgG antibodies in a subject, and methods of vaccinating a subject against a respiratory viral infection. More specifically, these methods use glycated chitosan polymers.

### BACKGROUND OF THE INVENTION

Chitin is a linear homopolymer composed of N-acetylglucosamine units joined by beta 1→4 glycosidic bonds. Chitosan is a derivative of chitin, a compound usually isolated from the shells of some crustaceans such as crab, lobster and shrimp. Due to the presence of quaternary ammonium salt groups, chitosan exhibits positive charges in physiological environments, and a large number of active functional groups on the polymer make chitosan easy to be structurally and chemically modified to produce a number of therapeutically useful features. Thus, chitin, chitosan (partially deacetylated chitin) and their derivatives possess interesting chemical and biological properties that have led to a varied and expanding number of industrial and medical applications. Glycated chitosan, described in U.S. Pat. No. 5,747,475 (“Chitosan-Derived Biomaterials”) is one such chitosan derivative. Glycated chitosan polymers are also disclosed in PCT Published Application No. WO 2013/109732 and U.S. Published Patent Application No. 2018/0312611.

Infectious diseases are currently one of the main causes of human death. The emergence of coronavirus disease-19 (COVID-19) has brought a significant health crisis, with high rates of morbidity and mortality recorded everywhere in the world. Seven coronaviruses infect humans currently, which include severe disease-causing strains such as acute respiratory syndrome coronavirus (SARS-CoV), Middle East respiratory syndrome coronavirus (MERS-CoV), and the COVID-19 causing virus named SARS-Cov-2. However, in addition to coronavirus family, there are a large number of respiratory viruses, such as Influenza, respiratory syncytial virus, rhinovirus, adenovirus and others that cause significant illness worldwide. Accordingly, new therapies for treatment of respiratory viral infections are needed, especially ones that exhibit excellent safety profiles and low cost.

### SUMMARY OF THE INVENTION

A method of treating, preventing and/or inhibiting one or more of the symptoms of a respiratory viral infection in a subject is provided, wherein the method comprises administering to the subject a therapeutically effective amount of a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

A method of reducing morbidity and/or mortality of a respiratory viral infection in a subject is also provided, wherein the method comprises administering to the subject a therapeutically effective amount of a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

Further provided is a method of inducing an innate immune response in mucosa of a subject, wherein the method comprises administering to the mucosa of the subject a therapeutically effective amount of a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

A method of generating mucosal secretory IgA antibodies and/or neutralizing serum IgG antibodies in a subject is provided, wherein the method comprises administering to the subject a therapeutically effective amount of a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

A method of vaccinating a subject against a respiratory viral infection is additionally provided, wherein the method comprises administering to the subject a therapeutically effective amount of a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

A vaccine composition for treatment and/or prevention of a respiratory viral infection is provided, the composition comprising one or more respiratory viral antigens and a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

Methods of reducing morbidity and/or mortality of a respiratory viral infection in a subject, methods of inducing an innate immune response in mucosa of a subject, methods of generating mucosal secretory IgA antibodies and/or neutralizing serum IgG antibodies in a subject, and methods of vaccinating a subject against a respiratory viral infection are also provided, wherein these methods comprise administering to the subject the vaccine composition disclosed herein.

### BRIEF DESCRIPTION OF THE DRAWINGS

FIG. 1 depicts survival curves post Sars-Cov-2 challenge in mice inoculated with GC and viral antigens compared to the ones inoculated with PBS and viral antigens.

FIG. 2 depicts antibody levels after intranasal SARS-Cov2 challenge.

FIG. 3 depicts levels of CD8+CD44+ T cells after subcutaneous vaccine.

FIG. 4 shows GC's ability to retain antigen locally and its ability to activate different pathways.

FIG. 5 shows that GC drives strong antigen specific humoral immune responses following a two-dose vaccine regimen.

FIG. 6 depicts that GC drives strong cellular responses following a two-dose vaccine regimen.

FIG. 7 depicts humoral immune responses (IgG and IgA levels) and cellular responses (CD8+ and CD4+ cells) following intranasal vaccination in mice.

FIG. 8 shows that combining GC with the trimeric spike protein and the nucleocapsid protein increases the influx of T cells into the lung following intranasal vaccination in mice.

FIG. 9 depicts lung cellularity (CD8+ T cells, CD4+ T cells and B cells) following a two-dose vaccine regimen.

FIG. 10 shows that spike and GC and nucleocapsid and GC vaccinated animals are resistant to SARS-CoV-2 viral challenge.

FIG. 11 shows lung cellularity of mice vaccinated with GC with nucleocapsid protein (5 μg) or spike protein (5 μg).

FIG. 12 depicts survival curves for intranasally vaccinated mice.

FIG. 13 depicts lung histopathology of mice vaccinated with GC, GC in combination with spike protein and nucleocapsid protein and controls.

### DEFINITIONS

The terms “treatment”, “treating” and the like are used herein to generally mean obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease or in delaying the disease. “Treatment” or “treating” or the like as used herein covers any treatment of a disease or symptom in a mammal, and includes: (a) preventing the disease or symptom from occurring in a subject who may be predisposed to acquiring the disease or symptom but has not yet been diagnosed as having it; (b) inhibiting the disease or symptom, i.e., arresting its development; or (c) relieving the disease, i.e., causing regression of the disease. Inhibiting or preventing a symptom means that an improvement is observed in the subject with respect to symptoms associated with the underlying disease, notwithstanding that the subject may still be afflicted with the underlying disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pies, horses, goats, and the like), etc.

It is noted that as used herein and in the appended claims, the singular forms “a,” “an,” and “the” include plural referents unless the context clearly dictates otherwise. Thus, for example, reference to “a viral antigen” includes a multitude of such antigens and reference to “a respiratory virus” includes reference to any of respiratory viruses known to those skilled in the art, and so forth.

The term “vaccine,” as used herein, includes any composition containing an immunogenic determinant, which stimulates the immune system such that it can better respond to subsequent infections. A vaccine usually contains an immunogenic determinant, e.g., an antigen, and an adjuvant, the adjuvant serving to non-specifically enhance the immune response to that immunogenic determinant. Currently produced vaccines predominantly activate the humoral immune system, i.e., the antibody dependent immune response. Other vaccines focus on activating the cell-mediated immune system including cytotoxic T lymphocytes, which are capable of killing targeted pathogens.

The term “adjuvant”, as used herein, refers to compounds that can be added to vaccines to stimulate immune responses against antigens, independent of the mechanism through which such stimulation is achieved. Adjuvants can enhance the immunogenicity of highly purified or recombinant antigens. Adjuvants can reduce the amount of antigen or the number of immunizations needed to protective immunity. For example, adjuvants can activate antibody-secreting B cells to produce a higher number of antibodies. Alternatively, adjuvants can act as a depot for an antigen, present the antigen over a longer period of time, which could help maximize the immune response and provide a longer-lasting protection. Adjuvants can also be used to enhance the efficacy of a vaccine by helping to modify the immune response to particular types of immune system cells, for example, by activating T cells instead of antibody-secreting B cells depending on the purpose of the vaccine.

As used herein, the term “administering,” refers to the delivery of a composition as disclosed herein into a subject by a method or route which results in at least partial delivery of the agent to the subject. The compositions disclosed herein can be administered by any appropriate route which results in an effective treatment in the subject, for example by intravenous, intranasal, intramuscular or subcutaneous route or can be administered by inhalation or nebulization.

By the terms “effective amount” and “therapeutically effective amount” of a composition or composition component is meant a sufficient amount of the composition or component, alone or in a combination, to provide the desired effect, such as prevention of disease or inhibition of one or more of the symptoms of disease, for which the composition or composition component is being administered.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable sub-combination. All combinations of the embodiments pertaining to the invention are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### DETAILED DESCRIPTION OF THE INVENTION

The present disclosure provides a number of methods related to treatment and prevention of respiratory viral infections. These methods use glycated chitosan (GC) polymers, which are linear homopolymers of deacetylated chitin (i.e., chitosan), wherein chitosan has a number of otherwise free amino groups bonded to a carbonyl group of a reducing monosaccharide or oligosaccharide.

The present disclosure provides methods of treating, preventing and/or inhibiting one or more of the symptoms of a respiratory viral infection in a subject, wherein the method comprises administering to the subject a therapeutically effective amount of a glycated chitosan (GC) polymer described herein. One or more symptoms of the respiratory viral infection that are inhibited by administration of the GC polymers include but are not limited to: fever and/or chills, cough, lethargy, muscle or body aches, sore throat, loss of taste and/or smell, congestion and/or runny nose, headache, nausea and/or vomiting, fatigue, rash and diarrhea. These symptoms can be fully inhibited or reduced. By way of example and not of limitation, administration of GC polymers described herein can result in prevention of fever, reduction of time that the fever lasts, or reduction in the intensity of the fever (e.g., prevention of a high temperature).

Provided herein is also a method of reducing morbidity and/or mortality of a respiratory viral infection in a subject by administering to the subject a therapeutically effective amount of a glycated chitosan (GC) polymer. Morbidity and mortality can be calculated as is standard in the medical field, and in some instances, morbidity and/or mortality are reduced by at least 10%, 20%, 30%, 40% or 50%.

The present disclosure provides a method of inducing an innate immune response in mucosa of a subject by administering to the subject a therapeutically effective amount of a glycated chitosan (GC) polymer. Non-limiting examples of mucosa include nasal, oral and lung mucosa. In some embodiments, the present methods induce an innate immune response in a nasal mucosa, and/or oral mucosa, and/or lung mucosa. The GC polymer can be administered in any manner that will result in induction of the innate immune response in a patient's nasal mucosa, such as by intranasal administration, inhalation or nebulization.

A method of generating mucosal secretory IgA antibodies and/or neutralizing serum IgG antibodies in a subject is also provided. The method comprises administering to the subject a therapeutically effective amount of a glycated chitosan (GC) polymer. The GC polymer can be administered in any manner that will result in generation of secretory IgA antibodies in mucosal tissues of a patient, such as by intranasal administration, inhalation or nebulization.

A method of vaccinating a subject against a respiratory viral infection is also provided. The method comprises administering to the subject a therapeutically effective amount of a glycated chitosan (GC) polymer. For purposes of vaccination, the GC polymer can be formulated as a vaccine with one or more pharmaceutically acceptable excipients. Furthermore, the GC polymer, when used in a method of vaccination, is typically administered prior to a viral infection or at its early onset; however, it can also be administered during the duration of the respiratory viral infection.

In any of these methods, a respiratory viral infection can be caused by any of the viruses that are capable of infecting respiratory tissues of a subject. In some instances, the respiratory viral infection is caused by any one of the viruses comprising rhinovirus, Influenza A, Influenza B, parainfluenza, coronavirus, respiratory syncytial virus (RSV) and adenovirus.

Rhinovirus is the most common viral infectious agent in humans and is the predominant cause of the common cold. The three species of rhinovirus (A, B, and C) include around 160 recognized types of human rhinovirus that differ according to their surface proteins (serotypes).

Influenza A virus causes influenza in birds and some mammals, and is the only species of the genus Alphainfluenzavirus of the virus family Orthomyxoviridae. Strains of all subtypes of influenza A virus have been isolated from wild birds, although disease is uncommon. Some isolates of influenza A virus cause severe disease both in domestic poultry and in humans. Influenza A viruses are negative-sense, single-stranded, segmented RNA viruses. The several subtypes are labeled according to an H number (for the type of hemagglutinin) and an N number (for the type of neuraminidase). There are 18 different known H antigens (H1 to H18) and 11 different known N antigens (N1 to N11).

Influenza B virus is the only species in the genus Betainfluenzavirus in the virus family Orthomyxoviridae. Influenza B virus is known only to infect humans and seals.

Human parainfluenza viruses (HPIVs) are the viruses that cause human parainfluenza. HPIVs are a paraphyletic group of four distinct single-stranded RNA viruses belonging to the Paramyxoviridae family. HPIVs remain the second main cause of hospitalization in children under 5 years of age suffering from a respiratory illness (only RSV causes more respiratory hospitalizations for this age group).

Coronaviruses are a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause Severe Acute Respiratory Syndrome (SARS), Middle East Respiratory Syndrome (MERS), and Coronavirus Disease 2019 (COVID-19). SARS is caused by severe acute respiratory syndrome coronavirus (SARS-CoV), MERS is caused by Middle East respiratory syndrome-related coronavirus (MERS-CoV), and Covid-19 is caused by severe acute respiratory syndrome coronavirus 2 (SARS-CoV2).

Respiratory syncytial virus (RSV), also called human respiratory syncytial virus (hRSV) and human orthopneumovirus, is a very common, contagious virus that causes infections of the respiratory tract. It is a negative-sense, single-stranded RNA virus, and its name is derived from the large syncytia that form when infected cells fuse together. While RSV is the single most common cause of respiratory hospitalization in infants, reinfection remains common throughout the lifetime, and it is an important pathogen in all age groups. Infection rates are typically higher during the cold winter months, causing bronchiolitis in infants, common colds in adults, and more serious respiratory illnesses such as pneumonia in the elderly and immunocompromised.

Adenoviruses (members of the family Adenoviridae) are medium-sized (90-100 nm), nonenveloped viruses with an icosahedral nucleocapsid containing a double stranded DNA genome. They have a broad range of vertebrate hosts; in humans, more than 50 distinct adenoviral serotypes have been found to cause a wide range of illnesses, from mild respiratory infections in young children (known as the common cold) to life-threatening multi-organ disease in people with a weakened immune system.

The GC polymer compositions disclosed herein can be used to treat respiratory viral infection caused by any of the viruses mentioned above. The GC polymer compositions are useful when the respiratory viral infection is caused by SARS-CoV2, MERS-CoV, Influenza H1N1 or Influenza H3N2. In some instances, the virus infections discussed herein can cause epidemics or pandemics.

A vaccine composition for treatment and/or prevention of a respiratory viral infection is also provided. The composition comprises one or more respiratory viral antigens and a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent. Any of the other GC polymers discussed herein can also be used. For example, the GC polymer can have molecular weight of about 250 kDaltons, a degree of glycation of free amino groups of about 5 percent, and a degree of deacetylation of a chitin parent of the GC polymer of about 80 percent.

Methods of reducing morbidity and/or mortality of a respiratory viral infection in a subject, methods of inducing an innate immune response in mucosa of a subject, methods of generating mucosal secretory IgA antibodies and/or neutralizing serum IgG antibodies in a subject, and methods of vaccinating a subject against a respiratory viral infection are also provided, wherein these methods comprise administering to the subject the vaccine composition disclosed herein. The considerations made for methods that only utilize GC polymer compositions apply to the vaccine compositions disclosed herein that comprise a respiratory viral antigen in addition to the GC polymer.

Exemplary GC polymers for use in the compositions and methods of the invention have been disclosed in U.S. Published Patent Application No. 2018/0312611 and PCT Published Application Nos. WO 2013/109732 and WO 2020/197578, all of which are incorporated herein by reference in their entirety; however, any glycated chitosans having a molecular weight of not more than 500,000 Daltons can be used in the methods and compositions of the invention.

The glycated chitosan polymers can have a molecular weight ranging from about 10,000 Daltons to about 500,000 Daltons, from about 50,000 to about 500,000 Daltons from about 75,000 to about 350,000 Daltons, from about 100,000 Daltons to about 300,000 Daltons, from about 150,000 Daltons to about 300,000 Daltons, or from about 200,000 Daltons to about 275,000 Daltons.

The glycated chitosan polymers can have a degree of glycation of free amino groups ranging from about one tenth of one percent to about 30 percent, from about one tenth of one percent to about 20 percent, from about one tenth of one percent to about 15 percent, from about one tenth of one percent to about 12.5 percent, from about one tenth of one percent to about 10 percent from about one tenth of one percent to about 7 percent, from about one tenth of one percent to about 5 percent, from about 2 percent to about 7 percent, or from about one tenth of one percent to about 2 percent.

The glycated chitosan polymers can have a degree of deacetylation of a chitin parent of the GC polymer ranging from about 70 percent to about 99 percent, or ranging from about 75 percent to about 85 percent.

For example, the glycated chitosan polymers can have a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

The GC polymers can have a molecular weight ranging from about 100,000 Daltons to about 300,000 Daltons, a degree of glycation of free amino groups ranging from about one tenth of one percent to about 30 percent, and a degree of deacetylation of a chitin parent of the GC polymer ranging from about 70 percent to about 99 percent.

The GC polymers can have a molecular weight ranging from about 200,000 Daltons to about 275,000 Daltons, a degree of glycation of free amino groups ranging from about one tenth of one percent to about 30 percent, and a degree of deacetylation of a chitin parent of the GC polymer ranging from about 70 percent to about 99 percent.

The glycated chitosans can have a molecular weight ranging from about 50,000 Daltons to about 400,000 Daltons, or from about 50,000 Daltons to about 350,000 Daltons, a degree of glycation of free amino groups ranging from about 2 percent to about 7 percent, and a degree of deacetylation of a chitin parent of the GC polymer ranging from about 70 percent to about 99 percent. These GC polymers can also have a molecular weight ranging from about 100,000 Daltons to about 300,000 Daltons, and further from about 200,000 Daltons to about 275,000 Daltons.

The glycated chitosans can have a molecular weight ranging from about 10,000 Daltons to about 500,000 Daltons, a degree of glycation of free amino groups ranging from about 2 percent to about 5 percent, and a degree of deacetylation of a chitin parent of the GC polymer ranging from about 70 percent to about 99 percent. Alternatively, such GC polymers can have a molecular weight from about 100,000 Daltons to about 300,000 Daltons, and further from about 200,000 Daltons to about 275,000 Daltons.

The GC polymers can have a molecular weight ranging from about 10,000 Daltons to about 500,000 Daltons, a degree of glycation of free amino groups ranging from one tenth of one percent to about 30 percent, and a degree of deacetylation of a chitin parent of the GC polymer ranging from about 75 percent to about 85 percent. Such GC polymers can also have a molecular weight from about 100,000 Daltons to about 300,000 Daltons, and further from about 200,000 Daltons to about 275,000 Daltons. Alternatively, these GC polymers can have a degree of glycation of free amino groups ranging from about one tenth of one percent to about 12.5 percent, ranging from about 2 percent to about 7 percent, and further ranging from about 2 percent to about 5 percent. The GC polymers can have a degree of deacetylation of a chitin parent of about 80 percent.

The GC polymers can have a molecular weight ranging from about 200,000 Daltons to about 275,000 Daltons, a degree of glycation of free amino groups of from about 2 percent to about 7 percent, and a degree of deacetylation of a chitin parent of the GC polymer ranging from about 75 percent to about 85 percent. Alternatively, the GC polymers have a molecular weight of about 250,000 Daltons, a degree of glycation of free amino groups of about 5 percent, and a degree of deacetylation of a chitin parent of the GC polymer of about 80 percent (this specific GC polymer is referred to in the Examples and Figures as GC).

Any respiratory viral antigens that are specific to a virus causing the respiratory infection can be used. When the respiratory virus is rhinovirus, antigens used for formulating a vaccine can be from the capsid proteins VP1-VP4, of which VP1-3 are surface exposed and represent the major targets for antibody responses. Rhinoviruses are challenging in terms of selecting antigens due to antigenic heterogeneity among more than 150 strains of human rhinovirus; however, shorter versions of VP1 and VP3 peptides have been relatively useful in generating neutralizing antibodies (Glanville and Johnston, Current Opinions in Virology, 2015, 11:83-88).

Influenza A antigens are well known and are used in vaccines that are commercially available for yearly flu vaccinations. By way of example, Influenza A and Influenza B antigens are commercially available from Sino Biological, which also lists all of the antigens used in vaccines from 2015-2021 (see, for example, sinobiological.com research web page regarding virus/influenza vaccine strains 2015-2021).

For human parainfluenza viruses (HPIV), antigens can include but are not limited to full length or truncated HN and/or F glycoproteins.

In cases of coronaviruses, antigens have been well described, and can include but are not limited to full length or truncated versions of the spike(S) protein, which is embedded in the viral surface envelope. A full length or truncated nucleocapsid (N) protein can also be used as a source of coronavirus antigens. Additionally, a combination of S and N protein antigens can also be used.

In cases of human respiratory synctytial virus, antigens can be selected from but are not limited to full length or truncated version of the fusion glycoprotein F. Additional targets can include small hydrophobic (SH) protein, which is a pentameric ion channel, and the putative attachment protein (G), which is a heavily O-glycosylated mucin-like glycoprotein.

With respect to human adenoviruses, vaccine antigen candidates include but are not limited to hexon and fiber antigens from Adenovirus type 5 (Ad5).

Additional antigens can be easily identified using known methods readily available to one of ordinary skill in the art. A skilled artisan can readily combine a viral antigen with the GC polymer disclosed herein to prepare a vaccine that can then be tested in a subject for efficacy. By way of example, efficacy can be measured by production of antibodies, which is a desirable property of a vaccine, and by a neutralizing antibody test as neutralizing antibodies are particularly beneficial in generating an immune response against a viral infection.

Additionally, the antigens are not limited to commercially available proteins. They can also include live attenuated pathogens, antigenic peptides, mRNA encoding for antigenic proteins or cells expressing the components of the pathogens.

Techniques used herein for antibody titer measurement include various known techniques such as radioimmunoassay (hereinafter referred to as “RIA”), solid-phase enzyme immunoassay (hereinafter referred to as “ELISA”), fluorescent antibody techniques, passive haemagglutination and so on, with ELISA being more preferred in terms of detection sensitivity, rapidity, accuracy, possible automation of operations, etc. For example, in an ELISA, antibody titer measurement can be accomplished by procedures described below. Firstly, purified or partially purified viral antigen is adsorbed on the solid phase surface such as 96-well plates for ELISA, and then contacted with a test sample (e.g., mouse serum from a test subject who has received a vaccine disclosed herein) for a period of time and under conditions sufficient to form antibody/antigen conjugates. After washing, an enzyme-labeled anti-mouse antibody is added as a secondary antibody and bound to the primary antibody in the serum that has been captured on the plate by the adsorbed antigen. After washing, a substrate for the enzyme is added, and changes in absorbance induced by color development based on substrate degradation are measured to thereby calculate the antibody titer.

There are many methods known in the art for measuring vaccine efficacy, and thus for purposes of the present disclosure determining whether a selected respiratory viral antigen is suitable for preparation of the disclosed vaccine comprising the respiratory viral antigen and a GC polymer. Briefly, if the vaccine shows efficacy, it means that the selected respiratory viral antigen is useful for combining it with a GC polymer to prepare a vaccine as disclosed herein.

One of the main and exemplary methods for measuring vaccine efficacy is a neutralizing antibody test. The neutralizing antibody test measures the ability of a subject's antibody to neutralize infectivity and protect cells from infection, and can be performed as is standard in the art. By way of example and not of limitation, it can be performed as briefly described below. A challenge dose of infectious virus is mixed with serial dilutions of a subject's serum after the subject has received one or more of the doses of the vaccine compositions disclosed herein. After e.g., a one-hour incubation, the mixture is inoculated on to a cell culture monolayer. Most often, the monolayer is overlayed with a semisolid medium to facilitate the production of virus-infected foci or plaques. After a defined incubation period the monolayers are fixed and stained, and the virus-induced plaques are counted. The endpoint is the dilution of the patient's serum that reduces plaque formation by 90%.

As antigens are peptides or full-length polypeptides, they can be synthesized using methods known in the art. Techniques for peptide synthesis can be either solid phase synthesis techniques or liquid phase synthesis techniques. Namely, a partial peptide or amino acids capable of constituting such a peptide are condensed with the remainder part and, if the product has protective groups, these protective groups are eliminated, whereby a desired peptide can be prepared. Procedures known for condensation and elimination of protective groups can be found in the following documents, by way of example: (i) M. Bodanszky and M. A. Ondetti, Peptide Synthesis, Interscience Publishers, New York (1966); and (ii) Schroeder and Luebke, The Peptide, Academic Press, New York (1965). After reaction, the peptide can be purified and isolated by standard procedures for purification, such as solvent extraction, distillation, column chromatography, liquid chromatography and recrystallization, which are used in combination. If the peptide thus obtained is in a free form, it can be converted into an appropriate salt form in a known manner. Conversely, if the peptide is obtained in a salt form, it can be converted into a free form in a known manner.

Peptides and polypeptides used as antigens can also be produced using recombinant expression in bacterial or mammalian cells. These techniques are well known and are detailed in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

An appropriate amount of a viral antigen and GC polymer for use in vaccines provided herein can readily be determined by combining varying quantities thereof, and determining a therapeutically effective amount by administering the vaccine to a test subject, and then reading out antibody titers, neutralizing antibody counts, and the like.

The vaccine or pharmaceutical composition provided herein can comprise a pharmaceutically acceptable excipient, a variety of which are known in the art and need not be discussed in detail herein. The terms “pharmaceutically acceptable excipient” and “pharmaceutically acceptable carrier” are used interchangeably herein. Pharmaceutically acceptable excipients have been amply described in a variety of publications, including, for example, “Remington: The Science and Practice of Pharmacy”, 19th Ed. (1995), or latest edition. Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7.th ed., Lippincott, Williams, & Wilkins; and Handbook of Pharmaceutical Excipients (2000) A. H. Kibbe et al., eds., 3rd ed. Amer. Pharmaceutical Assoc.

The compositions disclosed herein can comprise other components, such as pharmaceutical grades of mannitol, lactose, starch, magnesium stearate, sodium saccharin, talcum, cellulose, glucose, sucrose, magnesium, carbonate, and the like.

The compositions can contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

The compositions can be presented in unit-dose or multi-dose sealed containers, such as ampules and vials. Administration can be a single dose schedule or a multiple dose schedule. A primary dose schedule can be followed by a booster dose schedule. Suitable timing between priming and boosting can be routinely determined. By way of example, the GC polymers disclosed herein can be administered daily prior to exposure to a virus. For example, the GC polymers can be formulated for intranasal administration or inhalation so that a patient can administer them daily, e.g., during a flu season. The GC polymers of the present invention can also be administered during the infection with a respiratory virus. Vaccines of the present invention comprising the GC polymers can be administered on a two dose schedule, e.g., as an initial dose and a boosted dose administered 2 weeks, 4 weeks, 6 weeks, 8 weeks, 10 weeks or 12 weeks following the initial dose. Other dosing schedules can readily be determined as is standard in the art.

The pharmaceutical compositions disclosed herein can be administered via the respiratory system of a subject. One example of such administration is intranasal. Suitable formulations for administration include but are not limited to nasal spray or nasal drops, administration by nebulization or by inhalation. A pharmaceutical composition described herein can be administered by a nebulizer such as a jet nebulizer or an ultrasonic nebulizer.

Typically, in a jet nebulizer, a compressed air source is used to create a high-velocity air jet through an orifice. As the gas expands beyond the nozzle, a low-pressure region is created, which draws a composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer.

In an ultrasonic nebulizer, high-frequency electrical energy is used to create vibrational, mechanical energy, typically employing a piezoelectric transducer. This energy is transmitted to the composition creating an aerosol.

In a metered dose inhaler (MDI) or in other device that uses propellant, a propellant, a composition, and any excipients or other additives are contained in a canister as a mixture with a compressed gas. Actuation of the metering valve releases the mixture as an aerosol.

Pharmaceutical compositions for use with a metered-dose inhaler device will generally include a finely divided powder containing a composition of the disclosure as a suspension in a non-aqueous medium, for example, suspended in a propellant with the aid of a surfactant.

The propellant can be any conventional material employed for this purpose such as chlorofluorocarbon, a hydrochlorofluorocarbon, a hydrofluorocarbon, or a hydrocarbon including trichlorofluoromethane, dichlorodifluoromethane, dichlorotetrafluoroethanol and 1, 1, 1, 2-tetrafluoroethane, HFA-134a (hydrofluroalkane-134a), HFA-227 (hydrofluroalkane-227), or the like.

In other aspects, the compositions disclosed herein can be administered intramuscularly or subcutaneously.

The GC polymers disclosed herein can be prepared using any known methods available to one of ordinary skill in the art. For example, the GC polymers can be prepared via a reductive amination reaction involving the free amino groups of chitosan and the carbonyl groups of reducing monosaccharides and/or oligosaccharides. This reaction is a 2-step process, involving first the formation of an imine between the chitosan and the reducing sugar, followed by reduction of the imine to the amine using a wide range of reducing agents.

The reducing sugar, for example, can be D-galactose. Additionally, chitosan can be non-enzymatically glycated utilizing any of a number of the same or different reducing sugars, e.g., the same or different monosaccharides and/or oligosaccharides. Examples of such monosaccharide glycosylation agents are the more naturally occurring D-trioses, D-tetroses, D-pentoses, D-hexoses, D-heptoses, and the like, such as D-glucose, D-galactose, D-fructose, D-mannose, D-allose, D-altrose, D-idose, D-talose, D-fucose, D-arabinose, D-gulose, D-hammelose, D-lyxose, D-ribose, D-rhamnose, D-threose, D-xylose, D-psicose, D-sorbose, D-tagatose, D-glyceraldehyde, dihydroxyacetone, D-erythrose, D-threose, D-erythrulose, D-mannoheptulose, D-sedoheptulose and the like. Suitable oligosaccharides include the fructo-oligosaccharides (FOS), the galacto-oligosaccharides (GOS), the mannan-oligosaccharides (MOS) and the like.

The products of the first step of the reaction are mainly a mixture of Schiff bases (i.e. the carbon atom from the carbonyl group is now doubly bonded to the nitrogen from the free amine releasing one molecule of water) and Amadori products (i.e. the carbon atom of said carbonyl group is singly bonded to the nitrogen atom of said amino group while an adjacent carbon atom is double bonded to an oxygen atom). The products of the first step can be used as such or after the second step of the reaction, the stabilization by reduction with hydrides, such as boron-hydride reducing agents, for example NaBH4, NaBHsCN, NaBH(OAc)3, etc., or by exposure to hydrogen in the presence of suitable catalysts.

Solubilization of the starting material chitosan can be achieved by dissolution in aqueous acidic solutions, both organic and inorganic, leading to the formation of water soluble chitosonium salts by protonation of the free amino groups.

Modifications of the amino groups of chitosan include the introduction of chemical groups such as carboxymethyl, glyceryl, N-hydroxybutyl and others.

Glycation, i.e., non-enzymatic glycosylation of the free amino groups of chitosan, followed by stabilization by reduction, offers a useful approach for the preparation of various pharmaceutical compositions utilized herein.

The GC polymer described herein is in the form of a Schiff base (i.e. the carbon atom of the initial carbonyl group double bonded to the nitrogen atom of the amino group, also known as the imine functional group), an Amadori product (i.e. the carbon atom of the initial carbonyl group bonded to the nitrogen atom of said amino group by a single bond while an adjacent carbon atom is double bonded to an oxygen atom forming a ketone group), or in its reduced secondary amine or alcohol, respectively.

The GC polymer can include a carbonyl reactive group.

Various products obtained by chitosan glycation can be utilized as such or reacted with other natural or synthetic materials, e.g., reaction of aldehyde-containing derivatives of GC with substances containing two or more free amino groups, such as on the side chains of amino acids rich in lysine residues as in collagen, on hexosamine residues as in chitosan and deacetylated glycoconjugates, or on natural and synthetic diamines and polyamines. This is expected to generate crosslinking through Schiff base formation and subsequent rearrangements, condensation, dehydration, etc.

Stabilization of modified GC materials can be made by chemical reduction or by curing involving rearrangements, condensation or dehydration, either spontaneous or by incubation under various conditions of temperature, humidity and pressure. The chemistry of Amadori rearrangements, Schiff bases and the Leukart-Wallach reaction are detailed in The Merck Index, Ninth Edition (1976) pp. ONR-3, ONR-55 and ONR-80, Library of Congress Card No. 76-27231, the same being incorporated herein by reference. The chemistry of nucleophilic addition reactions as applicable to the invention is detailed in Chapter 19 of Morrison and Boyd, Organic Chemistry, Second Edition (eighth printing 1970), Library of Congress Card No. 66-25695, the same being incorporated herein by reference.

NMR tracings can be used to verify the bonding of the monosaccharides and/or oligosaccharides to the chitosan polymer, whereas chemical measurement of remaining free amino groups, such as via a ninhydrin reaction, can be used to assess the degree of glycation.

The GC polymers of the present disclosure can be purified using sterile filtration. By way of example and not of limitation, GC polymers can be sterile filtered using a 0.22-micron filter. Alternatively, GC polymers can be sterilized by autoclaving as is known in the art. Sterile filtration is preferred to avoid any unwanted alteration to the GC polymer that may occur at autoclaving temperatures.

### Example 1

The glycated chitosan used in these examples is referred to in the Figures and Examples as GC, and it has the following characteristics: a molecular weight of about 250,000 Daltons, a degree of glycation of free amino groups of about 5 percent, and a degree of deacetylation of a chitin parent of the GC polymer of about 80 percent. It was synthesized and sterile filtered as described in PCT Published Application WO 2013/109732. GC was combined with 100 μg of purified N (nucleocapsid) and S1 subunit of the spike(S) proteins to intranasally vaccinate mice transgenic for human angiotensis-converting enzyme 2 (hACE2) on C57BL/6 background. There were 4 experimental groups: 1) PBS control (6 mice), 2) GC (6 mice) 3) PBS+S1 subunit of the S protein+nucleocapsid (NC or N) protein (7 mice) and 4) GC+NC protein+S1 protein (7 mice). The mice were vaccinated twice (first inoculation at day 0, and the booster inoculation at week 4), followed by Sars-Cov-2 intranasal challenge at week 6 following the second vaccination (or week 10 from day 0). Mice were evaluated for clinical symptoms and general health over 30 days, and mouse survival was evaluated based on the inoculation received.

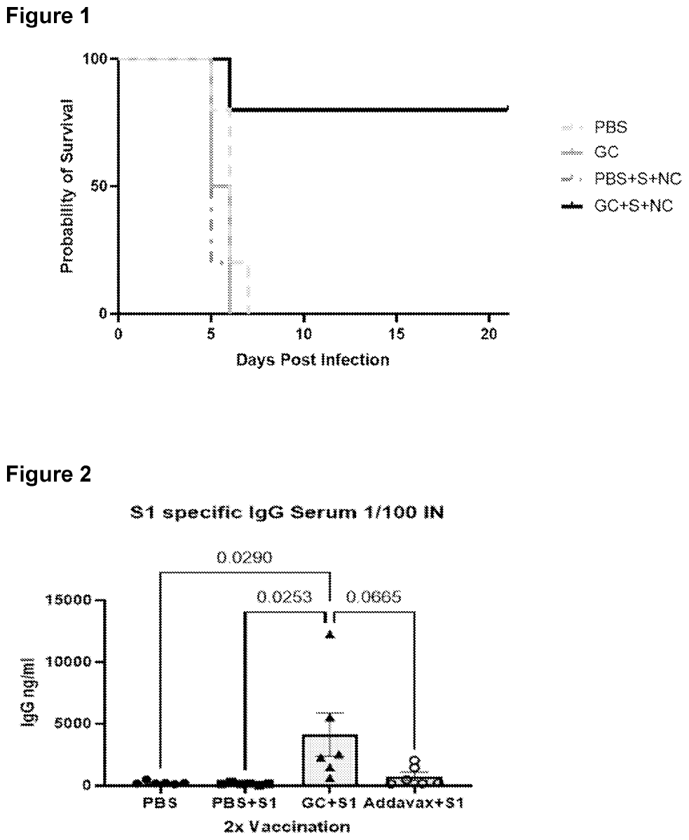

One mouse from each experimental group was euthanized at day 3 post viral challenge for harvesting tissues and their analysis. The mice were euthanized due to illness post viral challenge as follows: 1 mouse from group 1, 2 mice from group 2 and 4 mice from group 3 were euthanized at day 5 post Sars-Cov-2 challenge, 2 mice from group 1, 2 mice from group 2, 1 mouse from group 3 and 1 mouse from group 4 were euthanized at day 6, and 2 mice from group 1, 1 mouse from group 2 and 1 mouse from group 3 were euthanized at days 7-10 post Sars-Cov-2 challenge. One mouse from group 4 was euthanized at day 7 for tissue harvest and analysis, and at day 17 post viral challenge, 4 out of 7 mice from group 4 were still alive. The survival curves are shown in FIG. 1, and as can be seen from this figure, mice vaccinated with GC along with S protein and N protein antigens exhibited higher survival rates compared to mice in the other groups.

In addition to assessing survival, cardiac puncture was used to collect serum from mice, which will be used for antibody ELISAs to analyze to IgA, IgG1, and IgG2a. Mouse stool samples will be evaluated for viral shedding by virus isolation and PCR based assays. Viral titers in the blood, NALT, and lungs taken from samples prior to viral inoculation, 3 days post viral challenge, and at terminal harvest will be evaluated. Tracheal lavage samples obtained after inoculation and after viral challenge will be assessed for total IgA and IgG and N- and S-specific IgA/IgG. NALT and lung homogenate samples will be used for antibody and cytokine ELISAs. The spleens and lung draining lymph nodes obtained from euthanized mice will be used to prepare single cell suspensions, which will be incubated with N and S proteins to assess cytokine production by flow cytometry and ELISA (IFNγ and IL-4) and T cell specific responses.

### Example 2

hACE2 transgenic mice (n=6 per group) was inoculated in the nose with 2.5 μg of S1 protein (spike protein of SARs-CoV2 virus) with GC as described in Example 1, or PBS, or Addavax™ (a squalene-based oil-in-water nano-emulsion with a formulation similar to that of MF59® that has been licensed in Europe for adjuvanted flu vaccines) in a total of 20 μl. The vaccination was repeated 4 weeks later. Animals were harvested for analysis 7 days after the 2^{nd} vaccination. Serum was assessed for S1 specific IgG antibodies using indirect ELISA.

The following ELISA Protocol was used to detect antigen-specific serum antibodies. Nunc Maxisorp plates (Biolegend, cat #423501) were coated with antigen final concentration (10 μg/ml). The antigen was diluted to a final concentration of 10 mg/ml in binding buffer (0.1 M Na2HPO4, pH 9.0) add 100 μl per well. The plate was incubated overnight at 4° C., and wrapped in Saran wrap to prevent evaporation. Plates were washed 4× using wash buffer (1×PBS 0.05% Tween 20). Plates were blocked for 2 hours at room temperature with 1% w/v BSA in PBS/0.05% v/v Tween 20. 200 μl was used per well, and plates were washed before adding sample. For the standard controls, either IgG or IgA standards provided by the total ELISA kits were used. For Lung and NALT homogenate, undiluted or diluted samples were used. 50 μl were added to the plate containing 100 μl of PBS/0.05% Tween20. Plates were incubated overnight at 4° C. Plates were washed 4× with PBS/0.05% Tween. 50 μl per of detection antibody (1:250 dilution in PBS (1×)) was added to wells. The plate was sealed and incubated at room temperature for 2 hours on a shaker. The plate was washed 4× with 200 μl of wash buffer. 100 μl/well of substrate solution was added to each well, and plate was incubated at room temperature for 15 minutes. 100 μl of stop solution was added to each well (2N H2SO4). The plates were read at 450 nm. If wavelength subtraction was available, subtract values of 570 nm from those of 450 nm and the data were analyzed.

As can be seen from FIG. 2, in the group vaccinated twice intranasally with GC+S1, the mean level of S1 specific IgG in the serum was around 4500 ng/ml, which was significantly higher than S1 alone reconstituted in PBS (p=0.0253). Vaccination with Addavax™+S1 generated a much lower level of the same antibodies (<1000 ng/ml) (p=0.0665).

Thus, intranasal vaccination of GC with S1 protein was capable of eliciting a specific serum humoral response against the Spike protein S1 of the SARS-CoV2 virus. The level of antibody is triggered by GC was trending higher than nasal vaccine formulated with Addavax™.

### Example 3

hACE2 transgenic mice (n=6 per group) were inoculated subcutaneously in the back between the shoulder blades with 2.5 μg of S1 protein (spike protein of SARs-CoV2 virus) with GC as described in Example 1, or PBS, or Addavax™ in a total of 20 μl. The vaccination was repeated 4 weeks later. Animals were harvested for analysis 7 days after the 2^{nd} vaccination. Axillary lymph nodes were harvested and stained with antibodies, followed by flow cytometry to identify different immune populations.

Lymph nodes were isolated for analysis according to the following protocol. The cervical lymph nodes were isolated by removing the skin from the neck to expose the 4 lymph nodes underneath and removing surrounding fat. Each lymph node was placed in a 35×10 mm dish and pre-warmed. 1 ml Collagenase in RPMI (2% FCS) was added. Lymph node was digested for 20 minutes at 37° C. Lymph node and liquid were placed in a 100 μm cell strainer set in a 60 mm dish and crushed with the flat end of a 1 ml syringe plunger. 2 ml HBSS+2% FCS was added and run through the cell strainer. Liquid from 60 mm dish was placed into a 15 ml tube and spun at 1200 rpm for 3-5 minutes. Cells were resuspended in 1 ml HBSS+2% FBS and placed on ice until needed for further use.

### Trial Staining for Lung and Lymph Nodes on the Aurora

Panel to combine Lymphocytes and APCs

| Aurora | | | per 5e6 |
|---|---|---|---|
| Channel | Antibody | Flurochrome | cells |
| V1 | CD45 | BV421 | 0.8 ul |
| V3 | CD40 | Pacific Blue | 0.6 ul |
| V7 | Live/Dead | Ghost Dye Violet 510 | 0.8 ul |
| V8 | CD11b | BV570 | 0.6 ul |
| R1 | CD103 | APC | 0.8 ul |
| R2 | CD11c | AF647 | 0.6 ul |
| R4 | MHCII | RedFluor710 | 0.6 ul |
| V10 | CD86 | BV605 | 0.6 ul |
| V13 | Ly6G | SB702 | 0.5 ul |
| V15 | Ly6C | BV785 | 0.8 ul |
| YG1 | CD64 | PE | 0.6 ul |
| YG3 | F4/80 | PE/Dazzle | 0.5 ul |
| YG5 | B220 | PE-Cy5 | 0.5 ul |
| YG9 | CD19 | PE-Cy7 | 0.6 ul |
| B2 | CD24 | FITC | 0.6 ul |
| B9 | CD44 | PerCP-Cy5.5 | 0.5 ul |
| UV2 | CD8 | BUV395 | 0.5 ul |
| UV7 | CD3 | BUV496 | 0.8 ul |
| UV9 | CD4 | BUV563 | 0.5 ul |
| UV14 | CD62L | BUV737 | 0.7 ul |

Cells were counted and calculated in order to have ˜5e6 cells to stain. 100 μl of master mix was added to each tube and stained on ice for 15 minutes. 2 ml of FACs buffer was added to each tube and spun at 1200 rpm for 5 minutes. Supernatant was decanted and cells were resuspended in 100 μl of fixation buffer (brown bottle to clear bottle is a 1:3 ratio (i.e., for 32 samples 3.2 ml of fixation solution is needed). 1 ml of brown bottle solution and 3 ml of clear bottle solution were mixed thoroughly and aliquotted as 100 μl per tube. Each tube was fixed on ice for 20 minutes. Cells were washed with 2 ml of FACs buffer and spun at 1350 rpm for 5 minutes. Supernatant was decanted and cells resuspended in 300 μl of 1×PBS. Cells were stored at 4° C. or run flow.

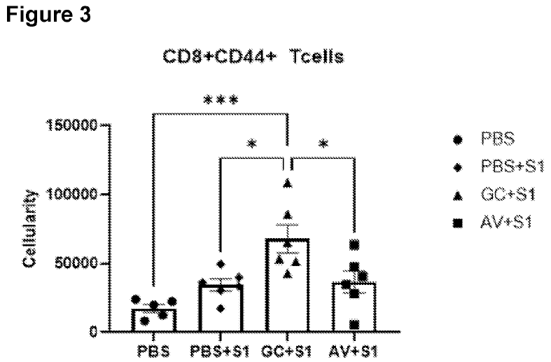

As can be seen from FIG. 3, in the group vaccinated twice subcutaneously with GC+S1, the mean level of CD8⁺CD44⁺ effector T cells was around 65000 cells per 2 axillary lymph nodes, which is close to ˜2-fold higher than S1 alone diluted in PBS (p<0.05) and >3-fold higher than the negative control (PBS with no S1). Vaccination with Addavax™+S1 only resulted in similar levels of CD8⁺CD44⁺ effector T cells as the S1 protein alone. Thus, subcutaneous vaccination of GC with Spike protein S1 of the SARs-CoV2 virus was capable of eliciting cellular immunity, and such effector T cell response was stronger than that induced by S1+Addavax™. Combined with example 2, GC was capable of eliciting both the cellular and humoral arms of immunity, which is important for anti-viral immunity and response against intracellular pathogens.

### Example 4

Transgenic mice expressing the human ACE2 (hACE2) gene from the human cytokeratin 18 (K18) promoter are inoculated intranasally with 1% GC in a final volume of 30 μl (15 μl per nostril) of phosphate buffered saline (PBS) solution. A control group is administered the same volume of PBS only. The mice are then challenged with Sars-Cov-2 virus 6 hours, 12 hours, 24 hours, and 48 hours following GC or PBS inoculation. In addition to assessing survival post Sars-Cov-2 challenge, stool samples are evaluated for viral shedding by virus isolation and PCR based assays. Viral titers in the blood, NALT, and lungs that are taken from samples prior to viral inoculation, 3 days post viral challenge, and at terminal harvest are evaluated. Cardiac puncture is used to collect serum from mice, which are used for antibody ELISAs to analyze to IgA, IgG1, and IgG2a. Tracheal lavage samples that are obtained after inoculation and after viral challenge are assessed for total IgA and IgG and N- and S-specific IgA/IgG. NALT and lung homogenate samples are used for antibody and cytokine ELISAs. The spleens and lung draining lymph nodes that are obtained from euthanized mice are used to prepare single cell suspensions, which are incubated with N and S proteins to assess cytokine production by flow cytometry and ELISA (IFNγ and IL-4) and T cell specific responses.

It is to be understood that this invention is not limited to particular embodiments described, as such may, of course, vary. It is also to be understood that the terminology used herein is for the purpose of describing particular embodiments only, and is not intended to be limiting, since the scope of the invention will be limited only by the appended claims.

### Example 5

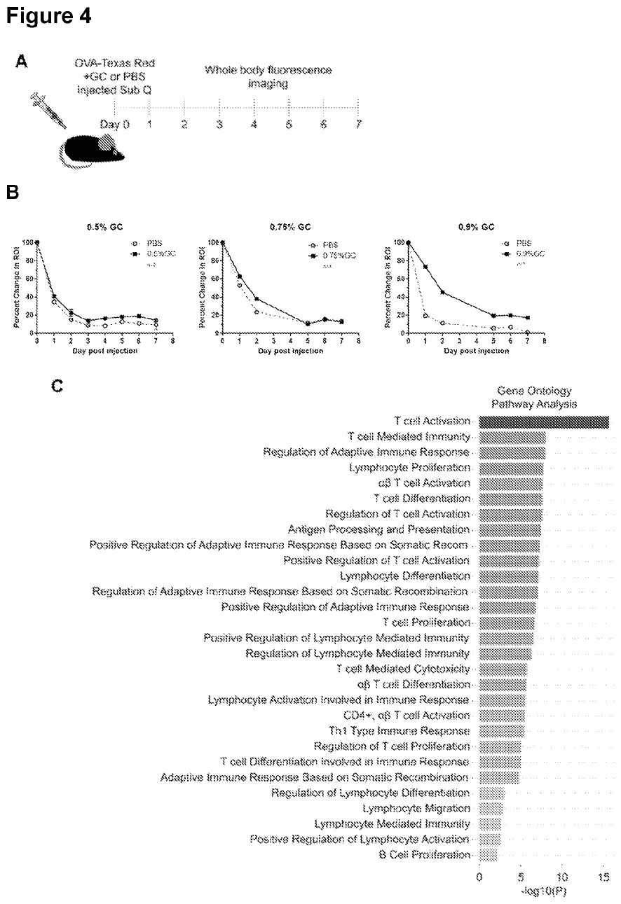

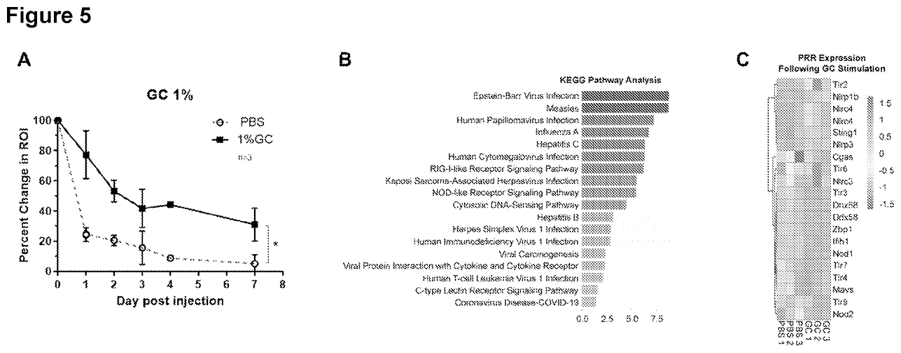

To determine if GC has the ability to retain antigen at the spot of injection, GC of different concentration (0.5, 0.75, 0.9, or 1%) was mixed with Texas-Red labeled ovalbumin (ova), administered to mice by local subcutaneous injection, and whole body fluorescent imaging was performed over a period of seven days (FIG. 4A-B). We observed that 1% GC was able to maintain ova at the site of injection significantly longer than PBS (FIG. 5A, FIG. 4B), Approximately 50% of the ova remained at the sight of injection in the 1% GC samples 3 days post injection, compared to about 20% with PBS. (FIG. 5A). This experiment demonstrates that GC can retain antigen at the site of injection.

### Example 6

Bulk RNA-sequencing of bone marrow derived dendritic cells (BMDCs) stimulated with GC (see “Materials and Methods” section) revealed that a variety of viral infection responses including Covid-19 were highly enriched (FIG. 5B). Several pathways involved in antigen processing and presentation, cytokine-cytokine receptor signaling, cell adhesion, cell-cell interaction, and antibody production networks were also upregulated (FIG. 4C). Moreover, a variety of pattern recognition receptors (PRRs), most notably a diverse array of nucleic acid PRRs was activated in response to GC (FIG. 5C). Taken together, these results suggest that GC could act as a potent adjuvant for recombinant protein vaccination as it activates multiple PRRs, drives the expression of genes required for T cell activation and differentiation, and triggers a variety of viral response pathways required to initiate antiviral immunity.

### Example 7

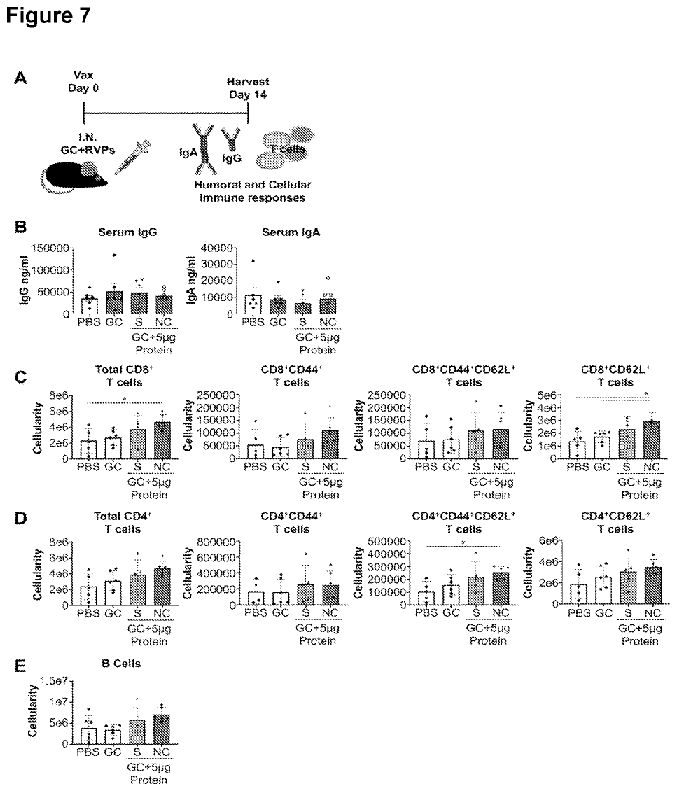

To examine the immune response generated after one vaccination with GC, 1% GC was combined with 5 μg of either recombinant SARS-CoV-2 trimeric spike protein(S) or the SARS-CoV-2 nucleocapsid protein (NC). C57BL/6 mice (8 weeks old) were vaccinated intranasally (I.N.) one time, and serum, lungs, and the cervical lymph nodes (cLN) were collected for analysis two weeks later (FIG. 7A). Total serum IgG and IgA were unchanged amongst the groups and S-specific antibodies were not detected at this time point (FIG. 7B). Lung cellularity and IgA levels were unchanged in the GC+S or GC+NC 1× vaccinated animals (data not shown). In the cLN, total CD8⁺ T cells and naïve CD8⁺CD62L⁺ T cells were increased in the GC+NC 1× vaccinated animals compared to PBS or GC only controls (FIG. 7C). For CD4⁺ T cells, the memory CD44⁺CD62L⁺CD4⁺ T cells were significantly increased in the GC+NC animals compared to PBS controls (FIG. 7D), and B cell numbers were unchanged (FIG. 7E). While one vaccination was able to increase lymph node cellularity, it was unable to generate S specific antibodies at a concentration that was detectable in this experiment. This suggests that one vaccination may not be enough to provide significant protection against viral challenge or that higher dosages of vaccines comprising viral antigen(s) and GC can be tested.

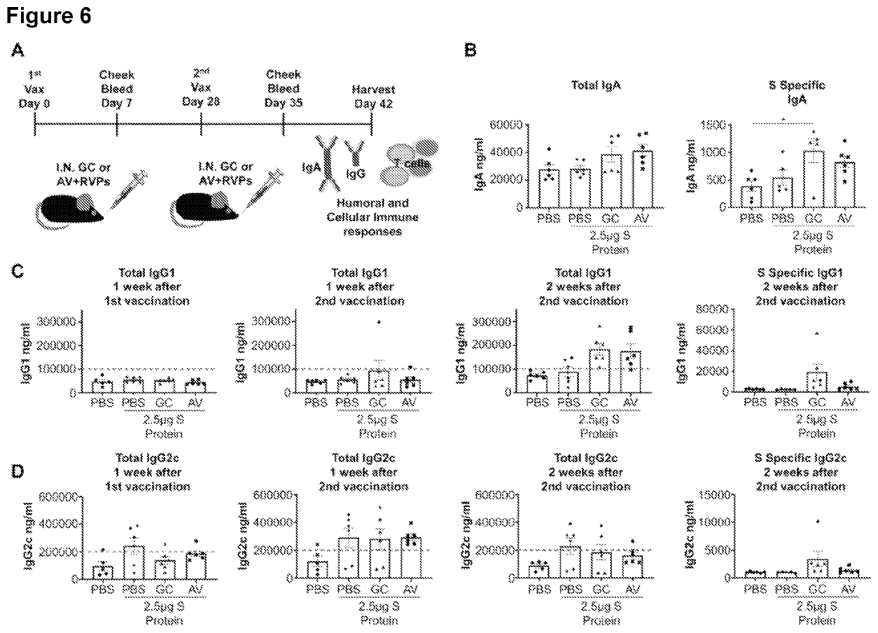

To that end, mice were next vaccinated with S intranasally (I.N.) twice, four weeks apart and examined the humoral and cellular immune responses two weeks later (FIG. 6A). The intranasal vaccination was performed because this is the route of infection for SARS-CoV-2 and no effective intranasal vaccines against SARS-CoV-2 have been developed so far. For adjuvant comparison purposes, MF-59 equivalent, Addavax™ (AV), was used because it is currently approved in human vaccines and has been demonstrated to enhance the mucosal immunogenicity of recombinant protein vaccination (Pulendran, B., S. Arunachalam, P. & O'Hagan, D. T. Emerging concepts in the science of vaccine adjuvants. Nature Reviews Drug Discovery (2021); Barchfeld, G. L. et al. The adjuvants MF59 and LT-K63 enhance the mucosal and systemic immunogenicity of subunit influenza vaccine administered intranasally in mice. Vaccine 17, 695-704 (1999)). PBS, 1% GC, or AV (15 μl) was mixed with 2.5 μg S protein and used for vaccination. Two weeks after 2^{nd} vaccination, left lobe lung homogenates were generated and total IgA and S-specific IgA were measured via antibody ELISA (FIG. 6B). While not significant by One-Way ANOVA, total IgA in both GC+S and AV+S vaccinated animals was trending higher, but S-specific IgA was significantly higher in the GC+S vaccinated animals compared to PBS controls (FIG. 6B). Total serum IgG1 and IgG2c antibodies were measured one week after the first vaccination, one week after the second vaccination, and two weeks after the second vaccination (FIG. 6C-D). One week after either the first vaccination or the second vaccination, there were no significant differences in total IgG1 among the four groups analyzed (FIG. 6C). Two weeks after the second vaccination, total IgG1 in both the GC+S and AV+S vaccinated animals was trending up but not significant by One-Way ANOVA (FIG. 6C). Interestingly, S-specific antibodies were not detected in the PBS samples, minimal detection was observed in the AV+S vaccinated animals, but GC+S vaccinated animals appeared to have an increase (FIG. 6C). One week after first vaccination IgG2c showed little change, while one week after second vaccination PBS+S, GC+S, and AV+S vaccinated animals experienced a trending increase (FIG. 6D). Interestingly, two weeks after the 2nd vaccination IgG2c levels returned to the levels observed one week after the first vaccination (FIG. 6D). Mirroring what was observed with S specific IgG1 (FIG. 6C), S specific IgG2c seemed to be most represented in the GC+S vaccinated group suggesting that GC drives a greater antigen specific antibody response compared to AV+S vaccination (FIG. 6D). Taken together, these data indicate that GC drives a satisfactory antigen specific humoral immune response that is comparable and possibly superior to a known vaccine adjuvant, AV.

### Example 8

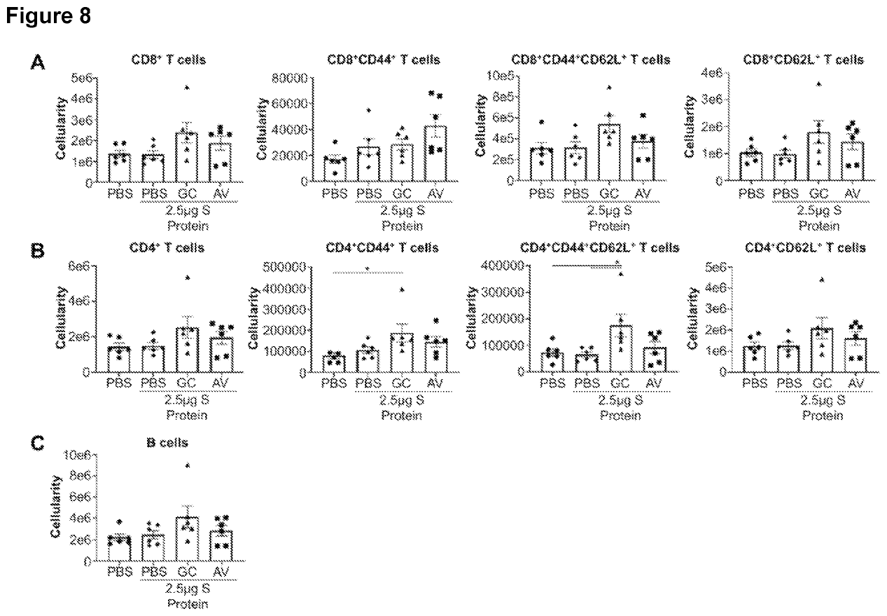

In addition to the humoral immune responses, the numbers of T and B cells residing within the cLN and the lungs following the same vaccination schedule described in FIG. 6A were examined. While not significant by one-way ANOVA, there was a trending increase in the total CD8⁺ T cells and specific CD8⁺ T cell subsets in the cLN of GC+S or AV+S vaccinated animals (FIG. 8A). Interestingly, GC+S vaccinated animals experienced a trending increase in CD8⁺CD44⁺CD62L⁺ and CD8⁺CD62L⁺ T cells while AV+S vaccinated animals demonstrated a trending increase in CD8⁺CD44⁺ and CD8⁺CD62L⁺ T cells (FIG. 8A). In contrast, CD4⁺CD44⁺ and CD4⁺CD44⁺CD62L⁺ were both significantly increased in the cLN of GC+S vaccinated animals but not in AV+S vaccinated animals (FIG. 8B). B cells were not significantly affected in any of experimental groups (FIG. 8C).

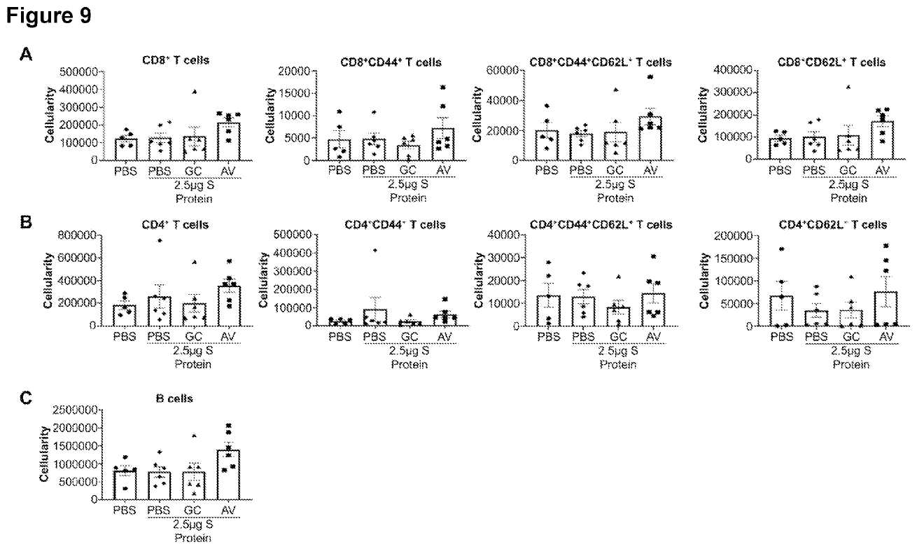

Next, the lung cellularity following a two-dose vaccine regimen was examined. GC+S vaccinated animals did not experience an increase of CD8⁺ T cells, CD4⁺ T cells, or B cells (FIG. 9A-C). However, AV+S vaccinated animals appeared to have a trending increase in CD8⁺ T cells and CD8⁺ T cell subsets (FIG. 9A). Total CD4⁺ T cells were trending up in AV+S vaccinated animals, but this was not reflected in the three CD4⁺ T cell subsets examined (FIG. 9B). Furthermore, B cells numbers also had a trending increase in AV+S vaccinated animals, which was not observed in the other 3 groups. Overall, the T cell cellularity within the lung following a two-dose I.N. vaccination regime resulted in minimal changes.

### Example 9

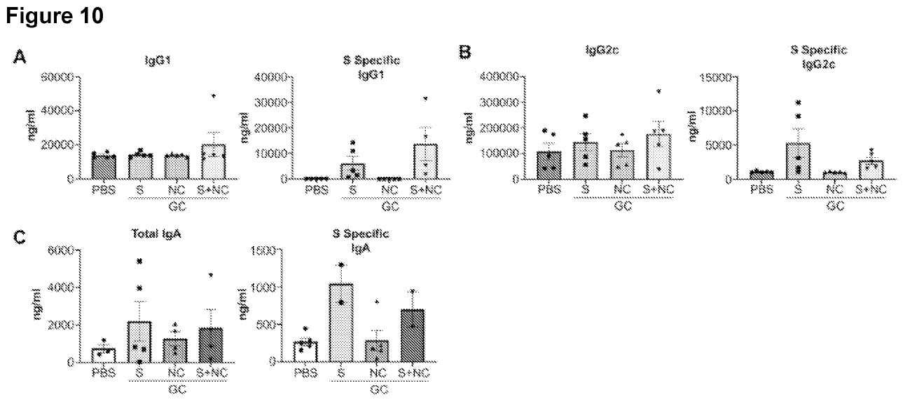

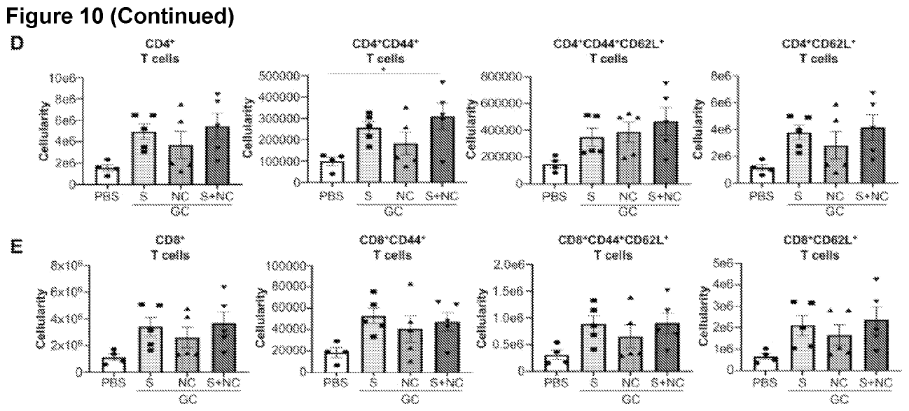

To examine whether the addition of the NC protein could increase the robustness of recombinant protein vaccine response, mice were vaccinated 2 times, 4 weeks apart, with either GC+5 μg S, GC+5 μg NC, or GC+2.5 μg S+2.5 μg NC. NC protein is one of the most abundant proteins in SARS-CoV-2 infected patients, highly immunogenic, highly conserved with a much slower mutation rate than the S protein and drives robust T and B cell response (Oliveira, S. C., de Magalhães, M. T. Q. & Homan, E. J. Frontiers in Immunology 11 (2020) Smits, V. A. J. et al. Biochemical and Biophysical Research Communications 543, 45-49 (2021); Lee, E. et al. J Virol 95 (2021); Lineburg, K. E. et al. Immunity 54, 1055-1065.e1055 (2021)). Two weeks after second vaccination, the serum, lungs, and cLNs were examined for antibody and cellular responses. In the serum, total IgG1 and IgG2c antibody levels were examined and it was found that they were largely unchanged in the GC+S, GC+NC, and GC+S+NC vaccinated animals compared to the PBS controls (FIG. 10A-B). However, when S specific IgG1 and IgG2c were examined, it was found that GC+S+NC, which used 2.5 μg S protein, made equivalent S specific IgG1 and IgG2c compared to 5 μg S+GC vaccinated animals (FIG. 10A-B). A similar trend was observed in the lungs with total IgA and S specific IgA (FIG. 10C), with GC+S and GC+S+NC making equivalent amounts of S specific IgA.

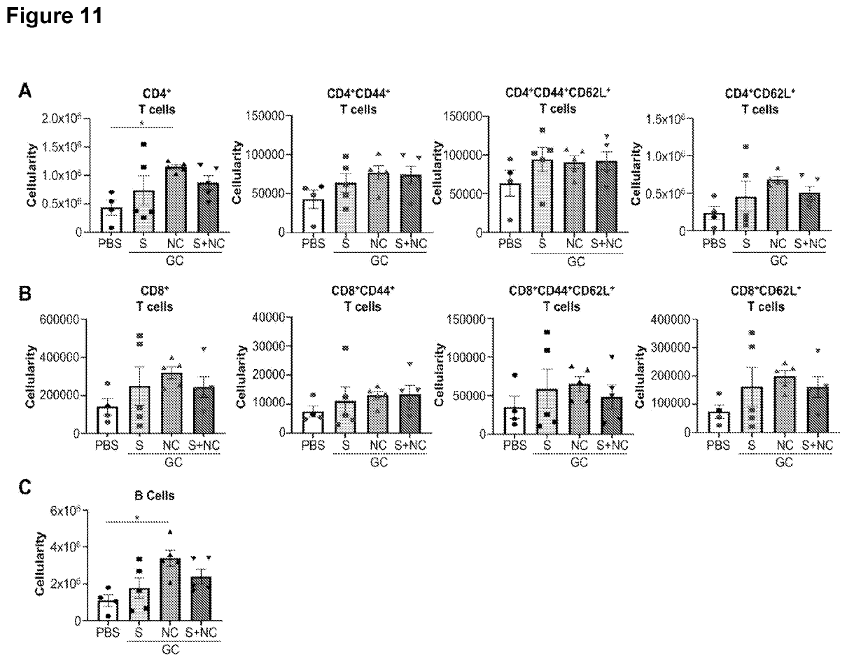

In the cLNs of the GC+S, GC+NC, and GC+S+NC vaccinated animals, trending increases in total and CD4⁺ and CD8⁺ T cell subsets (FIG. 10D-E) were observed, with only the CD4⁺CD44⁺ T cells being significantly increased in GC+S+NC vaccinated animals (FIG. 10D). In the lungs, either using 5 μg of S or NC protein, instead of 2.5 μg of S which was used in FIGS. 6 and 8, resulted in a trending increase in total CD4⁺ and CD8⁺ T cells and their subsets and B cells (FIG. 11A-C). Combining 2.5 μg of S and 2.5 μg of NC with GC resulted in an equivalent trending increase suggesting that combining the two proteins results in a synergistic yet more diverse immune response because it is targeting two separate SARS-CoV-2 proteins. For this reason, the S+NC combination was used next for viral challenge, as described below.

### Example 10

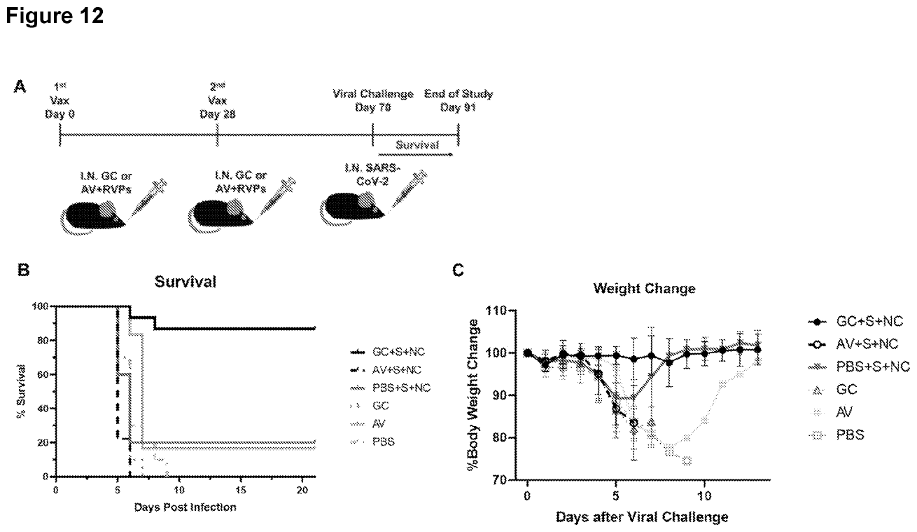

For viral challenge experiments, C57BI/6 transgenic mice expressing the human angiotensin 1-converting enzyme 2 (ACE2) receptor under control of the cytokeratin-18 (K18) gene promotor (K18-hACE2 mice) (McCray, P. B., Jr. et al., J Virol 81, 813-821 (2007) were vaccinated using the two-dose regimen described in the FIG. 6A. Six weeks after the second vaccination, animals were challenged intranasally with SARS-CoV-2 and survival was monitored for 21 days (FIG. 12A). Six groups were compared for the viral challenge: PBS, PBS+S+NC, GC, GC+S+NC, AV, or AV+S+NC. Within the first seven days following viral challenge, all mice in the PBS, and the GC alone vaccinated groups either died or had to be removed from the study due to severe health complications (FIG. 12B-C). One of six animals in the AV alone group survived, while two of ten PBS+S+NC vaccinated animals survived. Unexpectedly, all nine of the AV+S+NC vaccinated animals either died or had to be removed from the study due to a severe decline in health (FIG. 12B-C). In the GC+S+NC vaccinated group, 13 of the 15 vaccinated animals never experienced weight loss and survived the study without obvious complications (FIG. 12B-C). The two animals that did not survive, were removed for other health reasons, without experiencing dramatic weight loss.

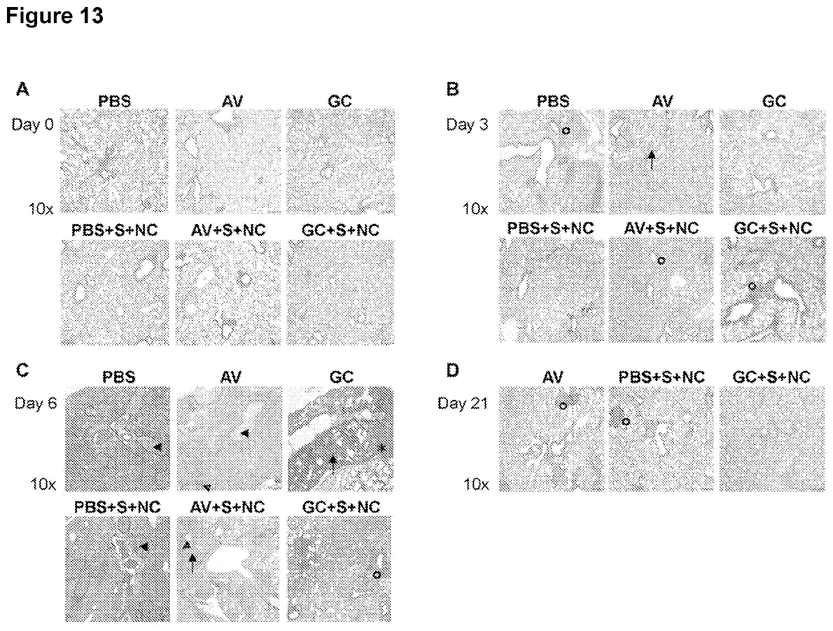

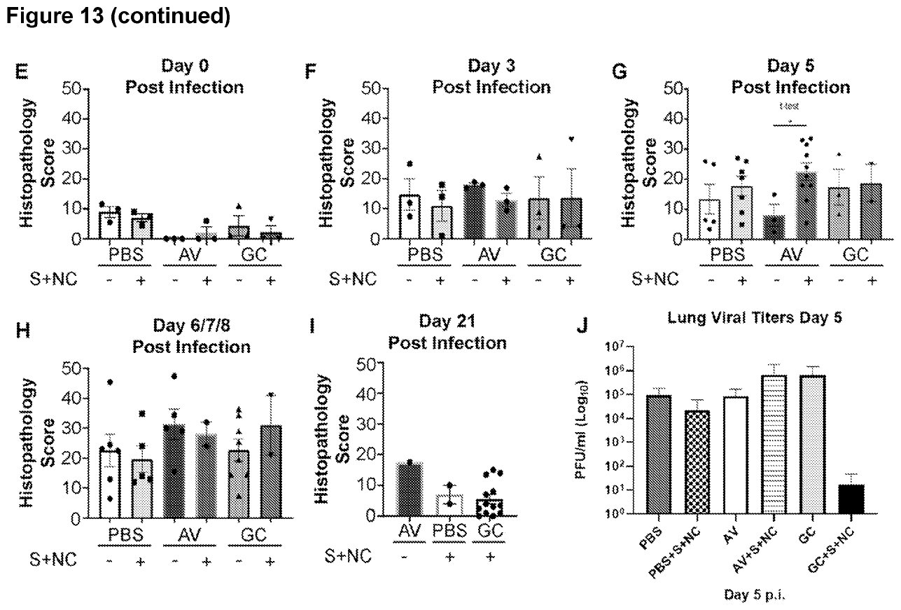

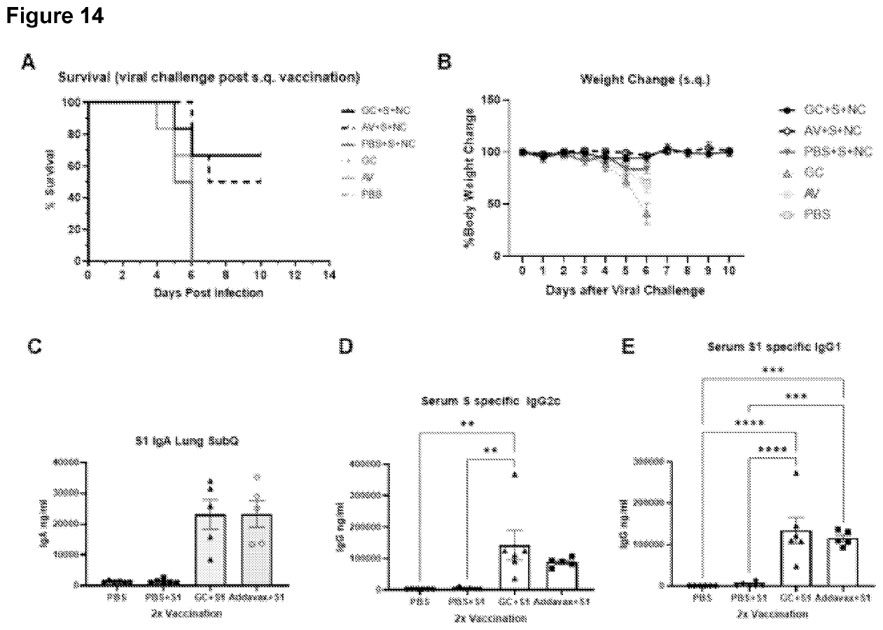

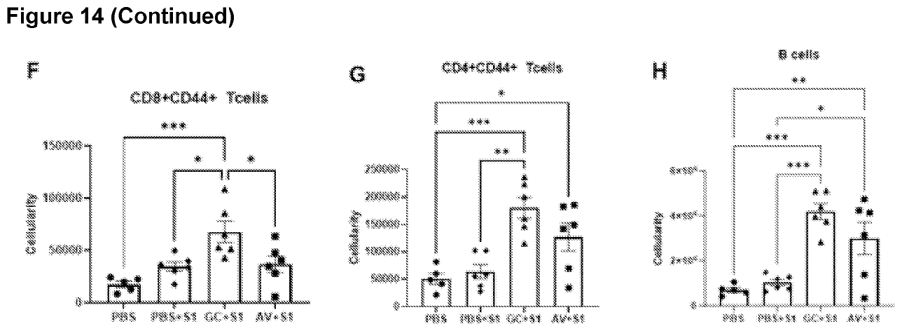

The histopathology of the lungs from the six groups of vaccinated animals was performed at days 0, 3, 6, and 21 post-infection is currently being analyzed. Interim pathology data shows that on day 6 post-infection, the PBS, GC, and PBS+S+NC groups exhibited the most severe changes (FIG. 13C). Moderate to large amounts of mucus admixed with inflammatory cells were observed and expanding the bronchioles (bronchiectasia, FIG. 13C, black arrows). There were also moderate numbers of lymphocytes, neutrophils, and macrophages in the interstitium and extending into the alveoli (FIG. 13C). While the GC+S+NC vaccinated animals had lung tissue containing peribronchiolar and perivascular inflammation six days after viral challenge, there was a complete absence of bronchiectasia (FIGS. 13C-D). The histopathology scoring is reported in FIGS. 13 E-I. In a separate cohort of animals where the lungs were harvested 5 days after viral challenge, the viral titer was markedly lower in the GC+S+NC vaccinated group (FIG. 13J).

When the same formulation of vaccine was injected into the mice subcutaneously under the same schedule, GC+S+NC provided greater than 60% protection against a lethal SARs-CoV2 challenge (FIG. 14A, B), similar to the examples with intranasal administration of GC+S1+NC vaccine (FIG. 12). In a separate cohort where the animals were vaccinated with GC+S1, elevated levels of S1 specific IgA in lung (FIG. 14C), S1 specific IgG1 (FIG. 14D) and IgG2c (FIG. 14E) in serum were detected compared to PBS+S1 controls 2 weeks after the second vaccination. They were comparable to the titers induced by Addavax™, which is the equivalent of the adjuvant MF59 approved for use in influenza vaccines. At the same time point, B cells in the draining lymph were found in a higher abundance after GC+S1 vaccination, indicating that GC drives a humoral response by inducing B-cell proliferation and class switching. Furthermore, GC+S1 vaccines were able to induce a statistically significant higher number of CD8+CD44+ effector T cells (FIG. 14F) than Addavax™+S1 and a trending increase in CD4+CD44+ effector T cells (FIG. 14G), showing superiority of GC in inducing cellular immunity when compared to Addavax™. As most adjuvants, like Addavax™, are skewed toward favoring a humoral response rather than a cellular response, GC poses an improvement because many pathogens, like viruses, require a mixed humoral and cellular immune response for the most effective and efficient clearance.

The above Examples utilized the following materials and methods.

### Materials and Methods

Animals: All animals studies were approved by the Oklahoma Medical Research Foundation, University of Oklahoma, Oklahoma State University, and IACUC. C57BL/6 were purchased from Jackson Laboratories (Stock number: 000664). Wild type C57BL/6 mice were used for the studies to analyze the cellular and humoral response to vaccination using either glycated chitosan (GC) and/or Addavax™ (InvivoGen, cat #vac-adx-10). B6.Cg-Tg (K18-ACE2) 2Prlmn/J were purchased from Jackson Laboratories (Stock number: 034860) and were used in the vaccination studies that were then challenged with SARS-CoV-2 to test the efficacy of recombinant protein/adjuvant intranasal vaccination.

ELISA: Total IgA (Cat #88-50450-88), IgG1 (Cat #88-50410-86), and IgG2c (Cat #88-50670-22) ELISA kits were purchased from ThermoFisher Scientific and were used according to manufactures instructions for the serum. Lung samples were diluted 1/1000 for analysis. For analysis of Trimeric Spike specific antibodies, Nunc MaxiSorp ELISA plates (Biolegend, Cat #423501) were coated with 10 μg/ml trimeric spike protein overnight at 4° C. Serum samples were diluted 1/1000 while lung samples were diluted 1/100. After incubation, the plates were developed using the reagents provided in the Total Ig ELISA kits. Standards for total IgA, IgG1, and IgG2c were used to generate standard curves and calculate the concentration of spike specific antibodies. Plates were read using the Bioteck Synergy H1 hybrid plate reader. Samples were analyzed and graphed using the GraphPad Prism Software. One-way AVOVA was used to determine statistical significance between the groups being compared.

Vaccination and Immune Cell Isolation: Recombinant SARS-CoV-2 proteins were purchased from R&D systems. For vaccination, either 5 μg or 2.5 μg of the trimeric spike protein (R&D Systems, cat #10549-CV-100) and the nucleocapsid protein (R&D Systems, cat #10474-CV-050), both resuspended in PBS, were mixed with 15 μl of 1% GC (Immunophotonics) or 15 μl of Addavax™ (InvivoGen, cat #vac-adx-10) mixed according to manufacturer instructions. Final volume to be delivered intranasal was 20 μl; 10 μl per nostril was delivered to male and female animals. Animals were vaccinated two times four weeks apart. The same formulation/dosage and vaccination schedule were used for subcutaneous injection experiments. Two weeks after first or second vaccination the cervical lymph nodes, lungs, serum, and spleen were isolated. Serum was isolated via cardiac puncture and acid-citrate-dextrose (ACD) solution was added to prevent coagulation. The left lobe was used for antibody isolation, while the remaining lobes were used for flow cytometry. Lungs were digested HEPES buffer containing Liberase™ 20 μg/ml (Roche, cat #5401127001) and DNase I (100 μg/ml) (Roche, cat #4536282001) for 25 minutes at 37° C. to isolate total leukocytes. Red blood cells were lysed using ACK lysis buffer and then resuspended in RPMI containing 10% FCS and Penicillin/Streptomycin until used for flow cytometry. Spleens and cervical lymph nodes were isolated and enzymatically in digested serum free RPMI containing 100 μg/ml Collagenase IV (Gibco, cat #17104019) and 20 μg/ml DNase I (Roche, cat #4536282001) for 20 minutes at 37° C. After digestion cells were washed in 1×HBSS, 5% fetal calf serum (FCS), and 5 mM EDTA and then for the spleens, the red blood cells were lysed using ACK lysis buffer. The cells were then resuspended in RPMI containing 10% FCS and Penicillin/Streptomycin until used for flow cytometry.

Flow Cytometry: The isolated leukocytes from the lungs, cervical lymph nodes, and spleens were stained with the following antibodies: CD45-BV421 (Biolegend, Cat #10314), CD40-Pacific Blue (Biolegend, Cat #124626), Ghost Dye-Violet-510 (Tonbo Biosciences, Cat #13-0870), CD11b-BV570 (Biolegend, Cat #101233), CD103-APC (Biolegend, Cat #121414), CD11c-AF647 (Biolegend, Cat #117312), MHCII-RedFluor710 (Tonbo Biosciences, Cat #80-5321), CD86-BV605 (Biolegend, Cat #105037), Ly6G-SB702 (Invitrogen, Cat #67-9668-82), Ly6C-BV785 (Biolegend, Cat #128041), CD64-PE (Biolegend, Cat #139304), F4/80 PE/Dazzle (Biolegend, Cat #123146), B220-PE-Cy5 (Biolegend, Cat #103210), CD19-PE-Cy7 (Invitrogen, Cat #25-0193-82), CD24-FITC (Biolegend, Cat #101806), CD44-PerCP-Cy5.5 (Tonbo Biosciences, Cat #65-0441), CD8-BUV395 (BD Horizon, Cat #565968), CD3-BUV496 (BD Optibuild, Cat #741117), CD4-BUV563 (BD Horizon, Cat #612923), CD62L-BUV737 (BD Horizon, Cat #612833). Briefly, 5e6 cells were stained with antibodies and viability on ice for 20 minutes in FACs buffer. Cells were washed and then fixed using Fixation Buffer (BD, Cat #554655) according to manufacturer's instructions. The cells were then washed and resuspended in 1×PBS and then analyzed on the Cytek Aurora spectral flow cytometer. Flow cytometry data was then analyzed using FlowJo software version 10.7 and then graphed using GraphPad Prism. One-way AVOVA was used to determine statistical significance between the groups being compared.

Lung homogenate: At the time of isolation, the left lung lobe was isolated and snap frozen in liquid nitrogen and stored in the −80° C. freezer until further use. To generate lung homogenate for antibody ELISA, lung tissues were thawed on ice and then resuspended in 0.5 ml of T-PER, Tissue Protein Extraction Reagent (ThermoScientific, cat #78510) containing Complete Mini Protease Inhibitor Cocktail (Roche, cat #11836170001). Tissues were homogenized using an electric handheld tissue homogenizer and were stored at −80° C. until used for ELISA.

Viral Challenge: B6.Cg-Tg (K18-ACE2) 2Prlmn/J were purchased from Jackson Laboratories (Stock number: 034860) and were used for viral challenge experiments. The animals were bred in-house according to IACUC approved protocols, and male and female mice heterozygous for the K18-hACE2 transgene were used for vaccination and viral challenge experiments. Animals were vaccinated as described above.

Immunohistochemistry and Histology Scoring: Organs were isolated from infected and uninfected mice, infused with 500 μl of 10% formalin, and placed in a 10% formalin container. Tissues for histopathology were processed routinely for 5 μm hematoxylin and eosin (H&E) stained sections. The lung sections were scored blindly by two board-certified veterinary pathologists and averages were obtained for each mouse lung. The interstitium, alveoli, bronchioles, and vessels of the lungs were evaluated for various parameters such as inflammation, edema, fibrin, necrosis, syncytial cells, hemorrhages, ectasia, vasculitis, and thrombi on the scale of 0-4:0, no lesions; 1, minimal (1 to 10% of lobes affected); 2, mild (11 to 40%); 3, moderate (41-70%); and 4, severe (71 to 100%). The cumulative score from the 24 parameters examined were graphed using GraphPad Prism. One-way AVOVA was used to determine statistical significance between the groups being compared.

BMDC Activation. BMDCs were stimulated with GC or 2′3-cGAMP (InvivoGen, Cat #tlrl-nacga23-1) for 0-24 hours prior to ELISA or flow cytometry. About 106 cells/ml BMDCs were plated into a non-tissue culture treated 96 well round bottom plate and then stimulated with 0.08-16 μg/ml GC. For 2′3-cGAMP stimulation, 200 ng of 2′3-cGAMP was first encapsulated into viromer green transfection reagent (Origene, Cat #TT100301) according to the manufacturer's instructions prior to incubation with BMDCs.

VX765 was purchased from InvivoGen (Cat #inh-vx765i-1) and used at a concentration of 10 μM. BMDCs were pre-incubated for 30 minutes with VX766 prior to the addition of GC. Bafitomycin A was purchased from InvivoGen (Cat #tlrl-baf1) and used at a concentration of 100 nM. BMDCs were pre-incubated for 30 minutes with bafilomycin A prior to the addition of GC. GSK 872 was purchased from Millipore Sigma (Cat #530389) and used at a concentration of 3 μM. BMDCs were pre-incubated for 45 minutes with GSK 872 prior to the addition of GC.

RNA-Sequencing. BMDCs were cultured for 24 hours, then harvested and stained with ghost dye BV510 for viability and CD11b APC-Cy7 and CD11c FITC. Live CD11b+CD11c+BMDCs were sorted on the BD FACS ARIA. RNA was isolated from the sorted BMDCs using the Quick-RNA microprep kit purchased from Zymo Research (Cat #R1050). RNA was isolated according to the manufacturer's protocol. For sequencing the 20M reads mRNA prep and sequencing service was performed for preparation of the RNA for NovaSeq PE150 reads on the NovaSeq6000.

Bioinformatics analysis (Quality control, read trimming, mapping to genome, and identification of DEGs). A quality check for the raw sequencing data was conducted using FastQC (v0.11.9) to detect common issues in RNA-Seq data. The reads were then trimmed with Trimmomatic (v0.39) to remove low quality bases51. The quality of the reads was re-evaluated with FastQC after this step to validate the quality improvements. The RNA-seq reads from each sample were mapped to the mouse mm10 genome assembly using the HISAT2. Samtools was used to manipulate the HISAT2 generated SAM files into BAM files52. FeatureCounts program from Subread package was used to count mapped RNAseq reads for genomic features53. DESeq2 was used for differential expressed genes (DEG) analysis based on the negative binomial distribution. The resulting P-values were adjusted using the Benjamini and Hochberg's approach for controlling the false discovery rate. Genes with an adjusted P-value (P adj)<0.05 as determined by DESeq2 were assigned as differentially expressed. Gene ontology (GO) analysis of DEG was performed using clusterProfiler54.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying figures shall be interpreted as illustrative and not in a limiting sense.

1. A method of treating, preventing and/or inhibiting one or more of the symptoms of a respiratory viral infection in a subject, and/or reducing morbidity and/or mortality of the respiratory viral infection in the subject, and/or inducing an innate immune response in mucosa of the subject, and/or generating mucosal secretory IgA antibodies and/or neutralizing serum IgG antibodies in the subject, and/or vaccinating the subject against the respiratory viral infection, the method comprising administering to the subject a therapeutically effective amount of a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent, wherein the administering to the subject treats, prevents and/or inhibits the one or more of the symptoms of the respiratory viral infection in the subject, and/or reduces morbidity and/or mortality of the respiratory viral infection in the subject, and/or induces the innate immune response in mucosa of the subject, and/or generates the mucosal secretory IgA antibodies and/or neutralizes the serum IgG antibodies in the subject, and/or vaccinates the subject against the respiratory viral infection.

2. The method of claim 1 wherein the administering to the subject reduces morbidity and/or mortality of the respiratory viral infection in the subject.

3. The method of claim 1, wherein the administering to the subject induces the innate immune response in mucosa of the subject.

4. The method of claim 1, wherein the administering to the subject generates mucosal secretory IgA antibodies and/or neutralizes serum IgG antibodies in the subject.

5. The method of claim 1, wherein the administering to the subject vaccinates the subject against the respiratory viral infection.

6. The method of claim 1, wherein the therapeutically effective amount of a glycated chitosan (GC) polymer is administered intranasally, intramuscularly or subcutaneously.

7. The method of claim 1, wherein the subject is a mammal.

8. The method of claim 7, wherein the mammal is a human.

9. The method of claim 1, wherein the respiratory viral infection is caused by rhinovirus, Influenza A, Influenza B, parainfluenza, coronavirus, respiratory syncytial virus (RSV) or adenovirus.

10. The method of claim 9, wherein the coronavirus is SARS-Cov-2 or MERS-Cov.

11. (canceled)

12. The method of claim 9, wherein the Influenza A is H1N1 or H3N2.

13.-14. (canceled)

15. The method of claim 1, wherein the GC polymer has a degree of deacetylation of a chitin parent of about 80 percent.

16. The method of claim 1, wherein the molecular weight of the glycated chitosan polymer ranges from about 50 kDa to about 350 kDa.

17. The method of claim 1, wherein the molecular weight of the glycated chitosan polymer ranges from about 100 kDa to about 300 kDa.

18. The method of claim 1, wherein the GC polymer has the degree of glycation of free amino groups ranging from about two percent to about seven percent.

19. The method of claim 1, wherein the GC polymer has the degree of glycation of free amino groups ranging from about two percent to about five percent, the molecular weight of about 250 kDa, and the degree of deacetylation of the chitin parent of about 80 percent.

20. The method of claim 1, wherein the therapeutically effective amount of the GC polymer is formulated as a nasal spray or nasal drops.

21. A vaccine composition for treatment and/or prevention of a respiratory viral infection, the composition comprising one or more respiratory viral antigens and a glycated chitosan (GC) polymer, wherein the GC polymer has a molecular weight ranging from about 10,000 Daltons to about 500,000 Daltons, a degree of glycation of free amino groups ranging from about one tenth of one percent to about 30 percent, and a degree of deacetylation of a chitin parent of the GC polymer ranging from about 70 percent to about 99 percent.

22.-36. (canceled)

37. A method of treating, preventing and/or inhibiting one or more of the symptoms of a respiratory viral infection in a subject, and/or reducing morbidity and/or mortality of the respiratory viral infection in the subject, and/or inducing an innate immune response in mucosa of the subject, and/or generating mucosal secretory IgA antibodies and/or neutralizing serum IgG antibodies in the subject, and/or vaccinating the subject against the respiratory viral infection, the method comprising administering to the subject the vaccine composition of claim 21.

38.-41. (canceled)

42. The method of claim 37, wherein the vaccine is administered intranasally.

43. The method of claim 1, wherein the administering to the subject treats, prevents and/or inhibits the one or more of the symptoms of the respiratory viral infection in the subject.
